(19) United States
(12) Patent Application Publication
(10) Pub. No.: US 2024/0316046 A1
(43) Pub. Date: Sep. 26, 2024

(54) ERK1/2 INHIBITOR COMBINATION THERAPY

(71) Applicants: Asana Biosciences, LLC, Bridgewater, NJ (US); The General Hospital Corporation, Boston, MA (US)

(72) Inventors: Sandeep GUPTA, Princeton, NJ (US); Louis DENIS, Princeton, NJ (US); Sanjeeva REDDY, Chester Springs, PA (US); Ryan B. CORCORAN, Weston, MA (US)

(21) Appl. No.: 18/570,818

(22) PCT Filed: Jun. 23, 2022
(86) PCT No.: PCT/US2022/034702

§ 371 Date: Dec. 15, 2023

Related U.S. Application Data
(60) Provisional application No. 63/214,764, filed on Jun. 24, 2021.

(51) Int. Cl. A61K 31/506; A61K 39/00; A61P 35/00

(52) U.S. Cl. CPC A61K 31/506; A61P 35/00; A61K 2039/505; A61K 2039/545

(57) ABSTRACT

The present invention relates generally to the use of an ERK1/2 inhibitor in combination with a B-Raf inhibitor that is encorafenib or dabrafenib for treating cancer, specifically solid tumors.

### CROSS-REFERENCE

This application claims the benefit of U.S. Provisional Application Ser. No. 63/214,764 filed Jun. 24, 2021, which is hereby incorporated by reference in its entirety.

### BACKGROUND OF THE INVENTION

ERK1 and ERK2 (collectively “ERK11/2”) are related protein-serine/threonine kinases that participate in, amongst others, the Ras-Raf-MEK-ERK signal transduction pathway, which is sometimes denoted as the mitogen-activated protein kinase (MAPK) pathway. This pathway is thought to play a central role in regulating a number of fundamental cellular processes including one or more of cell proliferation, survival, adhesion, cycle progression, migration, differentiation, metabolism, and transcription. The activation of the MAPK pathway has been reported in numerous tumor types including lung, colon, pancreatic, renal, and ovarian cancers. Accordingly, substances that could reduce activation could be of interest for possible treatments.

### SUMMARY OF THE INVENTION

ERK1/2 appear to be activated by MEK through phosphorylation of both a threonine and a tyrosine residue, namely at Tyr204/187 and Thr202/185. Once activated, ERK1/2 catalyze the phosphorylation of serine/threonine residues of more than 100 substrates and activate both cytosolic and nuclear proteins that are linked to cell growth, proliferation, survival, angiogenesis and differentiation, all hallmarks of the cancer phenotype. Thus it may be beneficial to target ERK 1 and ERK 2 to develop and use ERK1/2 inhibitors as a way to inhibit tumor growth.

Furthermore, an ERK inhibitor may have utility in combination with other kinase, for example MAPK, inhibitors. Recently, researchers reported that dual inhibition of MEK and ERK by small molecule inhibitors was synergistic and acted to overcome acquired resistance to MEK inhibitors. See Hatzivassiliou et al., ERK Inhibition Overcomes Acquired Resistance to MEK Inhibition, Mol. Cancer Ther. 2012, 11, 1143-1154.

In addition to ERK1/2, the RAS-MAPK signal transduction pathway includes the Raf family of proteins. The family includes composed of three related kinases (A-, B- and C-Raf) that act as downstream effectors of Ras. B-Raf, in particular is a serine/threonine protein kinase that activates the MAP kinase/ERK-signaling pathway. Constitutively active B-Raf mutants are commonly known to cause cancer by excessively signaling cells to grow. For example, activating B-Raf V600E kinase mutations occur in about 7% of human malignancies and about 50-60% of melanomas. The opportunity to target signal transduction pathways from multiple angles and potentially ameliorate feedback loops upstream of Ras via ERK1/2 and BRAF provides opportunities for developing methods that employ combination therapies.

The present embodiments disclosed herein generally relate to compositions and methods related to combination therapies to treat cancer utilizing an ERK1/2 inhibitor in conjunction with a B-Raf inhibitor that is encorafenib or dabrafenib while providing an unexpected degree of synergy.

In a first aspect, the present disclosure provides a method of treating cancer in a subject in need thereof, the method comprising: administering to the subject in need thereof a therapeutically effective amount of
(i) compound 1:

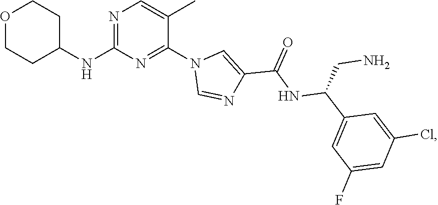 [SMILES: Cc1cnc(NC2CCOCC2)nc1-n1cnc(C(=O)N[C@H](CN)c2cc(F)cc(Cl)c2)c1]

or a pharmaceutically acceptable salt thereof, and
(ii) a BRAF inhibitor that is encorafenib or dabrafenib.

In a second aspect, the present disclosure provides a method of treating a cancer in a subject in need thereof, the method comprising administering to the subject in need thereof a therapeutically effective amount of
(i) compound 1:

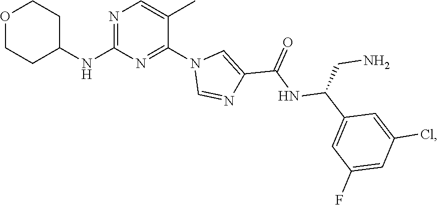 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) a BRAF inhibitor that is encorafenib or dabrafenib; and
(iii) panitumumab.

In a third aspect, the present disclosure provides a method of treating a cancer in a subject in need thereof, the method comprising administering to the subject in need thereof a therapeutically effective amount of
(i) compound 1:

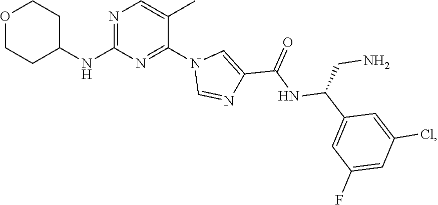 [SMILES: Cc1cnc(NC2CCOCC2)nc1-n1cnc(C(=O)N[C@H](CN)c2cc(F)cc(Cl)c2)c1]

or a pharmaceutically acceptable salt thereof; and
(ii) panitumumab.

In some embodiments, the BRAF inhibitor is encorafenib. In some embodiments, encorafenib is administered in an amount that is about 450 mg/day.

In some embodiments, the BRAF inhibitor is dabrafenib. In some embodiments, dabrafenib is administered in an amount that is about 150 mg/day.

In some embodiments, the method further comprises administering panitumumab. In some embodiments, panitumumab is administered in an amount that is about 6 mg/kg.

In some embodiments, the pharmaceutically acceptable salt of compound 1 is the mandelic acid salt.

In some embodiments, the cancer is a mitogen-activated protein kinase (MAPK) pathway driven cancer. In some embodiments, the cancer is aBRAF-driven cancer, HRAS-driven cancer, or aNRAS-driven cancer.

In some embodiments, the cancer comprises at least one cancer cell driven by deregulated ERK.

In some embodiments, the cancer has at least one mutation in RAS. In some embodiments, the cancer has at least one mutation in RAF. In some embodiments, the cancer has at least one mutation in MEK.

In some embodiments, the cancer has a G12C KRAS mutation. In some embodiments, the cancer has a G12C KRAS mutation. In some embodiments, the cancer has a G12D KRAS mutation. In some embodiments, the cancer has a G12S KRAS mutation. In some embodiments, the cancer has a G12V KRAS mutation. In some embodiments, the cancer has a G12C KRAS mutation. In some embodiments, the cancer has a G13D KRAS mutation. In some embodiments, the cancer has Q16H KRAS mutation. In some embodiments, the cancer has a Q16K KRAS mutation.

In some embodiments, the cancer has a Q61RNRAS mutation.

In some embodiments, the cancer is a BRAF V600E or V600K mutant tumor.

In some embodiments, the cancer is a MAPKm/MAPKi-naïve pan cancer.

In some embodiments, the cancer comprises one or more EGFR mutation selected from the group consisting of EGFR gene copy gain, EGFR gene amplification, chromosome 7 polysomy, L858R, exon 19 deletions/insertions, L861Q, G719C, G719S, G719A, V765A, T783A, exon 20 insertions, EGFR splice variants (Viii, Vvi, and Vii), A289D, A289T, A289V, G598A, G598V, T790M, and C797S.

In some embodiments, the cancer comprises one or more EGFR mutation selected from the group consisting of L858R, exon 19 deletion, and T790M.

In some embodiments, the cancer is a solid tumor. In some embodiments, the cancer is non-small cell lung cancer (NSCLC), melanoma, pancreatic cancer, salivary gland tumor, thyroid cancer, colorectal cancer (CRC), or esophageal cancer.

In some embodiments, the cancer is non-small cell lung cancer (NSCLC). In some embodiments, the NSCLC is an EGFR mutant NSCLC. In some embodiments, the NSCLC is a KRAS G12C mutant NSCLC. In some embodiments, the NSCLC is a KRAS G12D mutant NSCLC. In some embodiments, the NSCLC is a KRAS G12S mutant NSCLC. In some embodiments, the NSCLC is a KRAS G12V mutant NSCLC. In some embodiments, the NSCLC is a KRAS G13D mutant NSCLC. In some embodiments, the NSCLC is a KRAS Q61H mutant NSCLC. In some embodiments, the NSCLC is a KRAS Q61K mutant NS CLC.

In some embodiments, the NSCLC is aNRAS Q61R mutant NSCLC.

In some embodiments, the cancer is a MAPKm/MAPKi-naïve NSCLC.

In some embodiments, the cancer is BRAFi-treated V600 NSCLC.

In some embodiments, the cancer is KRAS-treated G12C NSCLC. In some embodiments, the cancer is KRAS-treated G12D NSCLC. In some embodiments, the cancer is KRAS-treated G12S NSCLC. In some embodiments, the cancer is KRAS-treated G12V NSCLC. In some embodiments, the cancer is KRAS-treated G13D NSCLC. In some embodiments, the cancer is KRAS-treated Q61H NSCLC. In some embodiments, the cancer is KRAS-treated Q61KNSCLC.

In some embodiments, the cancer is a NRAS-treated Q61RNSCLC.

In some embodiments, the cancer is pancreatic cancer. In some embodiments, the cancer is a MAPKm/MAPKi-naïve pancreatic cancer.

In some embodiments, the cancer is melanoma. In some embodiments, the melanoma is a BRAF V600E or V600K mutant tumor. In some embodiments, the cancer is a BRAFi-treated V600 melanoma.

In some embodiments, the cancer is salivary gland tumor.

In some embodiments, the cancer is thyroid cancer.

In some embodiments, the cancer is colorectal cancer (CRC). In some embodiments, the CRC is a BRAF V600E CRC.

In some embodiments, the CRC is a KRAS mutant CRC. In some embodiments, the CRC is a KRAS G12C mutant CRC. In some embodiments, the CRC is a KRAS G12D mutant CRC. In some embodiments, the CRC is a KRAS G12S mutant CRC. In some embodiments, the CRC is a KRAS G12V mutant CRC. In some embodiments, the CRC is a KRAS G13D mutant CRC. In some embodiments, the CRC is a KRAS Q61H mutant CRC. In some embodiments, the CRC is a KRAS Q61K mutant CRC.

In some embodiments, the CRC is a NRAS mutant CRC. In some embodiments, the CRC is a NRAS Q61R mutant CRC.

In some embodiments, the cancer is esophageal cancer.

In some embodiments, compound 1, or a pharmaceutically acceptable salt thereof, is administered in an amount that is between about 25 mg/day and about 300 mg/day. In some embodiments, compound 1, or a pharmaceutically acceptable salt thereof, is administered in an amount that is between 25 mg/day and 150 mg/day.

In some embodiments, compound 1, or a pharmaceutically acceptable salt thereof, is administered in an amount that is between about 25 mg/day, about 50 mg/day, about 75 mg/day, about 100 mg/day, about 150 mg/day, about 175 mg/day, about 200 mg/day, about 225 mg/day, or about 250 mg/day.

In some embodiments, compound 1, or a pharmaceutically acceptable salt thereof, is administered in an amount that is between about 25 mg/day, about 50 mg/day, about 100 mg/day, or about 150 mg/day.

In some embodiments, compound 1, or a pharmaceutically acceptable salt thereof, is administered once a day (QD). In some embodiments, compound 1, or a pharmaceutically acceptable salt thereof, is administered twice a day (BID). In some embodiments, compound 1, or a pharmaceutically acceptable salt thereof, is administered three times a day (TID).

In some embodiments, compound 1, or a pharmaceutically acceptable salt thereof, is administered once a week. In some embodiments, compound 1, or a pharmaceutically acceptable salt thereof, is administered twice a week.

In some embodiments, compound 1, or a pharmaceutically acceptable salt thereof, is administered in an amount that is between about 50 mg once a week and about 400 mg once a week. In some embodiments, compound 1, or a pharmaceutically acceptable salt thereof, is administered in an amount that is between about 50 mg twice a week and about 400 mg twice a week.

In some embodiments, compound 1, or a pharmaceutically acceptable salt thereof, is administered for at least one 28-day cycle. In some embodiments, compound 1, or a pharmaceutically acceptable salt thereof, is administered on day 1, day 8, day 15, and day 22 of a 28-day cycle. In some embodiments, compound 1, or a pharmaceutically acceptable salt thereof, is administered on day 1, day 8, day 15 of a 28-day cycle.

In some embodiments, compound 1, or a pharmaceutically acceptable salt thereof, is administered for at least one 21-day cycle.

In some embodiments, compound 1, or a pharmaceutically acceptable salt thereof, is administered orally.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows cell viability assay data for Compound 1 and encorafenib in RKO cells.

FIG. 1B shows cell viability assay data for Compound 1 and encorafenib in HT-29 cells.

FIG. 1C shows cell viability assay data for Compound 1 and encorafenib in WiDr cells.

FIG. 1D shows cell viability assay data for Compound 1 and encorafenib in MDST8 cells.

FIG. 1E shows cell viability assay data for Compound 1 and encorafenib in LIM2405 cells.

FIG. 2A shows in-vivo data for compound 1+encorafenib in encorafenib refractory-BRAF^{V600E} RKO CDX models.

FIG. 2B shows in-vivo data for compound 1+encorafenib in encorafenib refractory-BRAFV^{V600E}WiDr CDX models

FIG. 3A shows Western blot gels depicting phosphorylation of RSK (P-RSK) and ERK (P-ERK) using two BRAF V600E mutant CRC cell lines, RKO (top) and HT-29 (bottom), with encorafenib in combination with binimetinib.

FIG. 3B shows Western blot gels depicting phosphorylation of RSK (P-RSK) and ERK (P-ERK) using two BRAF V600E mutant CRC cell lines, RKO (top) and HT-29 (bottom), with compound 1 in combination with encorafenib.

FIG. 3C shows Western blot gels depicting phosphorylation of RSK (P-RSK) and ERK (P-ERK) using two BRAF V600E mutant CRC cell lines, RKO (top) and HT-29 (bottom), with LY3214996 in combination with encorafenib.

FIG. 3D shows Western blot gels depicting phosphorylation of RSK (P-RSK) and ERK (P-ERK) using two BRAF V600E mutant CRC cell lines, RKO (top) and HT-29 (bottom), with Ravoxertinib in combination with encorafenib.

### DETAILED DESCRIPTION OF THE INVENTION

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

As used herein, the term “therapeutic” means an agent utilized to treat, combat, ameliorate, prevent, or improve an unwanted condition or disease of a patient. In some embodiments, a therapeutic agent such as a compound 1 is directed to the treatment and/or the amelioration of cancers.

“Administering” when used in conjunction with a therapeutic means to administer a therapeutic systemically or locally, as directly into or onto a target tissue, or to administer a therapeutic to a patient whereby the therapeutic positively impacts the tissue to which it is targeted. Thus, as used herein, the term “administering,” when used in conjunction with a composition described herein, can include, but is not limited to, providing a composition into or onto the target tissue; providing a composition systemically to a patient by, e.g., oral administration whereby the therapeutic reaches the target tissue or cells. “Administering” a composition may be accomplished by injection, topical administration, and oral administration or by other methods alone or in combination with other known techniques.

The term “animal” as used herein includes, but is not limited to, humans and non-human vertebrates such as wild, domestic and farm animals. As used herein, the terms “patient,” “subject” and “individual” are intended to include living organisms in which certain conditions as described herein can occur. Examples include humans, monkeys, cows, sheep, goats, dogs, cats, mice, rats, and transgenic species thereof. In a preferred embodiment, the patient is a primate. In certain embodiments, the primate or subject is a human. In certain instances, the human is an adult. In certain instances, the human is child. In further instances, the human is under the age of 12 years. In certain instances, the human is elderly. In other instances, the human is 60 years of age or older. Other examples of subjects include experimental animals such as mice, rats, dogs, cats, goats, sheep, pigs, and cows. The experimental animal can be an animal model for a disorder, e.g., a transgenic mouse with hypertensive pathology.

By “pharmaceutically acceptable,” it is meant the carrier, diluent or excipient must be compatible with the other ingredients of the formulation and not deleterious to the recipient thereof.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology).

The terms “treat,” “treated,” “treatment,” or “treating” as used herein refers to both therapeutic treatment in some embodiments and prophylactic or preventative measures in other embodiments, wherein the object is to prevent or slow (lessen) an undesired physiological condition, disorder, or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition. As used herein, “treat,” “treated,” “treatment,” or “treating” includes prophylaxis in some embodiments.

The term “substantially the same as” as used herein, refers to a powder x-ray diffraction pattern or differential scanning calorimetry pattern that is non-identical to those depicted herein, but that falls within the limits of experimental error, when considered by one of ordinary skill in the art.

### Compound 1

### Disclosed herein is (S)-N-(2-amino-1-(3-chloro-5-fluorophenyl)ethyl)-1-(5-methyl-2-((tetrahydro-2H-pyran-4-yl)amino)pyrimidin-4-yl)-1H-imidazole-4-carboxamide

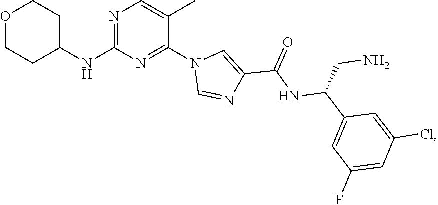 [SMILES: Cc1cnc(NC2CCOCC2)nc1-n1cnc(C(=O)N[C@H](CN)c2cc(F)cc(Cl)c2)c1]

or a pharmaceutically acceptable salt thereof.

In some embodiments, the salt of compound 1 is the mandelic acid salt. In some embodiments, the salt of compound 1 is the benzenesulfonic acid salt. In some embodiments, the salt of compound 1 is the hydrochloride salt. In some embodiments, the salt of compound 1 is the p-toluenesulfonic acid salt.

### Encorafenib

Encorafenib

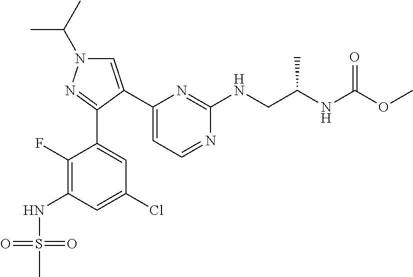 [SMILES: COC(=O)N[C@@H](C)CNc1nccc(-c2cn(C(C)C)nc2-c2cc(Cl)cc(NS(C)(=O)=O)c2F)n1]

is a drug for the treatment of certain melanomas. It is a small molecule BRAF inhibitor that targets key enzymes in the MAPK signaling pathway. This pathway occurs in many different cancers including melanoma and colorectal cancers. In June 2018, it was approved by the FDA in combination with binimetinib for the treatment of patients with unresectable or metastatic BRAF V600E or V600K mutation-positive melanoma. Encorafenib is sold as Braftovi® by Pfizer.

### Dabrafenib

Dabrafenib

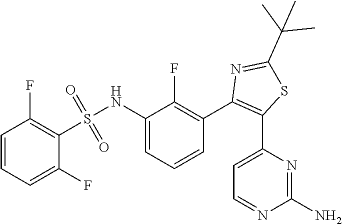 [SMILES: CC(C)(C)c1nc(-c2cccc(NS(=O)(=O)c3c(F)cccc3F)c2F)c(-c2ccnc(N)n2)s1]

is a medication for the treatment of cancers associated with a mutated version of the gene BRAF. Dabrafenib acts as an inhibitor of the associated enzyme B-Raf, which plays a role in the regulation of cell growth. Dabrafenib has clinical activity with a manageable safety profile in clinical trials of phase 1 and 2 in patients with BRAF (V600)-mutated metastatic melanoma. Dabrafenib is sold as Tafinlar® by Novartis.

### Panitumumab

Panitumumab, formerly ABX-EGF, is a fully human monoclonal antibody specific to the epidermal growth factor receptor (also known as EGF receptor, EGFR, ErbB-1 and HER1 in humans).

Panitumumab is an epidermal growth factor receptor (EGFR) antagonist indicated for the treatment of wild-type KRAS (exon 2) metastatic colorectal cancer (mCRC) as determined by an FDA-approved test for this use: In combination with FOLFOX for first-line treatment or as monotherapy following disease progression after prior treatment with fluoropyrimidine, oxaliplatin, and irinotecan-containing chemotherapy. Panitumumab is sold as Vectibix® by Amgen.

### Combinations

Disclosed herein is a method of treating cancer in a subject in need thereof, the method comprising: administering to the subject in need thereof a therapeutically effective amount of
(i) compound 1:

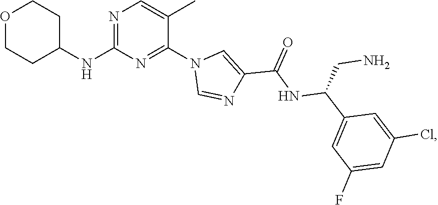 [SMILES: Cc1cnc(NC2CCOCC2)nc1-n1cnc(C(=O)N[C@H](CN)c2cc(F)cc(Cl)c2)c1]

or a pharmaceutically acceptable salt thereof; and
(ii) a BRAF inhibitor that is encorafenib or dabrafenib.

In some embodiments, compound 1 is combined with encorafenib. In some embodiments, compound 1 is combined with dabrafenib.

Disclosed herein is a method of treating cancer in a subject in need thereof, the method comprising: administering to the subject in need thereof a therapeutically effective amount of
(i) compound 1:

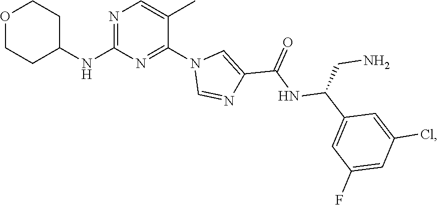 [SMILES: Cc1cnc(NC2CCOCC2)nc1-n1cnc(C(=O)N[C@H](CN)c2cc(F)cc(Cl)c2)c1]

or a pharmaceutically acceptable salt thereof; and
(ii) a BRAF inhibitor that is encorafenib.

Disclosed herein is a method of treating cancer in a subject in need thereof, the method comprising: administering to the subject in need thereof a therapeutically effective amount of
(i) compound 1:

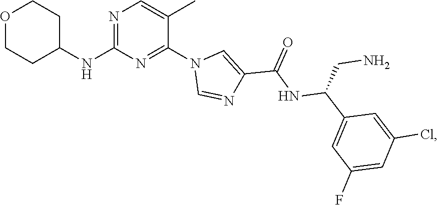 [SMILES: Cc1cnc(NC2CCOCC2)nc1-n1cnc(C(=O)N[C@H](CN)c2cc(F)cc(Cl)c2)c1]

or a pharmaceutically acceptable salt thereof; and
(ii) a BRAF inhibitor that is dabrafenib.

Disclosed herein is a method of treating a cancer in a subject in need thereof, the method comprising: administering to the subject in need thereof a therapeutically effective amount of
(i) compound 1:

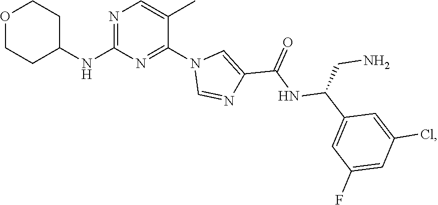 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) a BRAF inhibitor that is encorafenib or dabrafenib; and
(iii) panitumumab.

Disclosed herein is a method of treating a cancer in a subject in need thereof, the method comprising: administering to the subject in need thereof a therapeutically effective amount of
(i) compound 1:

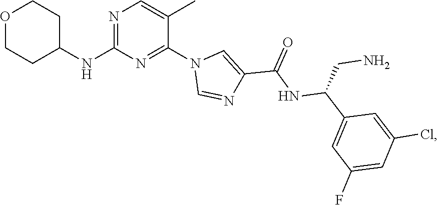 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) a BRAF inhibitor that is encorafenib; and
(iii) panitumumab.

Disclosed herein is a method of treating a cancer in a subject in need thereof, the method comprising: administering to the subject in need thereof a therapeutically effective amount of
(i) compound 1:

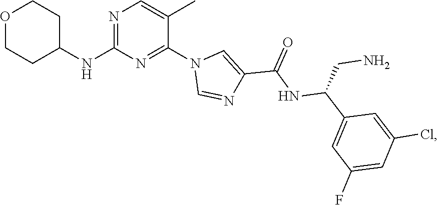 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) a BRAF inhibitor that is dabrafenib; and
(iii) panitumumab.

Disclosed herein is method of treating a cancer in a subject in need thereof, the method comprising: administering to the subject in need thereof a therapeutically effective amount of
(i) compound 1:

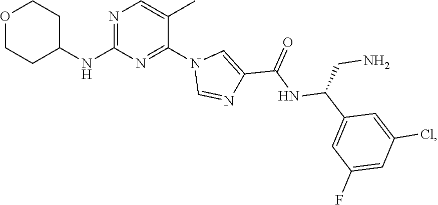 [SMILES: Cc1cnc(NC2CCOCC2)nc1-n1cnc(C(=O)N[C@H](CN)c2cc(F)cc(Cl)c2)c1]

or a pharmaceutically acceptable salt thereof; and
(ii) panitumumab.

### Cancers

Disclosed herein are methods of treating cancer using a combination disclosed herein.

“Cancer” refers to all types of cancer, neoplasm or malignant tumors found in mammals (e.g. humans), including, without limitation, leukemias, lymphomas, myelomas, carcinomas, and sarcomas. Exemplary cancers that may be treated with a compound or method provided herein include brain cancer, glioma, glioblastoma, neuroblastoma, prostate cancer, colorectal cancer, pancreatic cancer (such as pancreatic adenocarcinoma, PDAC), medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the blood, thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

In some embodiments, the cancer has a class 1 B-Raf mutation.

In some embodiments, the cancer harbors at least one of a EGFR, KRAS, BRAF (e.g., BRAF class III) and/or NF1 (e.g., loss of function) mutations.

In some embodiments, the mutant B-Raf comprises a V600 mutation. In some embodiments, the mutant of B-Raf comprises the mutation V600E. In some embodiments, the mutation is V600K. In some embodiments, the mutation is V600D. In some embodiments, the mutation is V600L. In some embodiments, the mutation is V600R. In some embodiments, the cancer is a BRAF V600E or V600K mutant tumor.

In some embodiments, the cancer is a mitogen-activated protein kinase (MAPK) pathway driven cancer.

In some embodiments, the cancer is a BRAF-driven cancer, HRAS-driven cancer, or aNRAS-driven cancer.

In some embodiments, the cancer comprises at least one cancer cell driven by deregulated ERK.

In some embodiments, the cancer has at least one mutation in RAS. In some embodiments, the cancer has at least one mutation in RAF. In some embodiments, the cancer has at least one mutation in MEK.

In some embodiments, the cancer has a G12C KRAS mutation. In some embodiments, the cancer has a G12D KRAS mutation. In some embodiments, the cancer has a G12S KRAS mutation. In some embodiments, the cancer has a G12V KRAS mutation. In some embodiments, the cancer has a G13D KRAS mutation. In some embodiments, the cancer has a Q16H KRAS mutation. In some embodiments, the cancer has a Q16K KRAS mutation. In some embodiments, the cancer has a Q61RNRAS mutation.

In some embodiments, the cancer is BRAF V600E or V600K mutant tumor.

In some embodiments, the cancer is a MAPKm/MAPKi-naïve pan cancer.

In some embodiments, the cancer comprises one or more EGFR mutation selected from the group consisting of EGFR gene copy gain, EGFR gene amplification, chromosome 7 polysomy, L858R, exon 19 deletions/insertions, L861Q, G719C, G719S, G719A, V765A, T783A, exon 20 insertions, EGFR splice variants (Viii, Vvi, and Vii), A289D, A289T, A289V, G598A, G598V, T790M, and C797S. In some embodiments, the cancer comprises one or more EGFR mutation selected from the group consisting of L858R, exon 19 deletion, and T790M.

In some embodiments, the cancer is a solid tumor. In some embodiments, the solid tumor is an advanced or a metastatic solid tumor.

In some embodiments, the cancer is non-small cell lung cancer (NSCLC), melanoma, pancreatic cancer, salivary gland tumor, thyroid cancer, colorectal cancer (CRC), or esophageal cancer.

In some embodiments, the cancer is non-small cell lung cancer (NSCLC). In some embodiments, the NSCLC is an EGFR mutant NSCLC. In some embodiments, the NSCLC is a KRAS G12C mutant NSCLC. In some embodiments, the NSCLC is a KRAS G12D mutant NSCLC. In some embodiments, the NSCLC is a KRAS G12S mutant NSCLC. In some embodiments, the NSCLC is a KRAS G12V mutant NSCLC. In some embodiments, the NSCLC is a KRAS G13D mutant NSCLC. In some embodiments, the NSCLC is a KRAS Q61H mutant NSCLC. In some embodiments, the NSCLC is a KRAS Q61K mutant NSCLC.

In some embodiments, the NSCLC is aNRAS Q61R mutant NSCLC. In some embodiments, the cancer is a MAPKm/MAPKi-naïve NSCLC. In some embodiments, the cancer is a BRAFi-treated V600 NSCLC. In some embodiments, the cancer is a KRAS-treated G12C NSCLC. In some embodiments, the cancer is a KRAS-treated G12D NSCLC. In some embodiments, the cancer is a KRAS-treated G12S NSCLC. In some embodiments, the cancer is a KRAS-treated G12V NSCLC. In some embodiments, the cancer is a KRAS-treated G13D NSCLC. In some embodiments, the cancer is a KRAS-treated Q61H NSCLC. In some embodiments, the cancer is a KRAS-treated Q61KNSCLC. In some embodiments, the cancer is a NRAS-treated Q61RNSCLC.

In some embodiments, the cancer is pancreatic cancer. In some embodiments, the cancer is a MAPKm/MAPKi-naïve pancreatic cancer. In some embodiments, the pancreatic cancer is pancreatic ductal adenocarcinoma (PDAC). In some embodiments, the PDAC is indicated by a KRAS G12V mutation.

In some embodiments, the cancer is melanoma. In some embodiments, the melanoma is a BRAF V600E or V600K mutant tumor. In some embodiments, the cancer is a BRAFi-treated V600 melanoma.

In some embodiments, the cancer is salivary gland tumor.

In some embodiments, the cancer is thyroid cancer.

In some embodiments, the cancer is colorectal cancer (CRC). In some embodiments, the CRC is a BRAF V600E CRC. In some embodiments, the CRC is a KRAS mutant CRC.

In some embodiments, the CRC is a KRAS G12C mutant CRC. In some embodiments, the CRC is a KRAS G12D mutant CRC. In some embodiments, the CRC is a KRAS G12S mutant CRC. In some embodiments, the CRC is a KRAS G12V mutant CRC. In some embodiments, the CRC is a KRAS G13D mutant CRC. In some embodiments, the CRC is a KRAS Q61H mutant CRC. In some embodiments, the CRC is a KRAS Q61K mutant CRC. In some embodiments, the CRC is a NRAS mutant CRC. In some embodiments, the CRC is aNRAS Q61R mutant CRC.

In some embodiments, the cancer is esophageal cancer.

In some embodiments, the cancer is colorectal cancer (CRC), pancreatic ductal adenocarcinoma (PDAC), cholangiocarcinoma cancer, appendiceal cancer, gastric cancer, esophageal cancer, non-small cell lung cancer (NSCLC), head and neck cancer, ovarian cancer, uterine cancer, acute myeloid leukemia (AML), or melanoma.

In some embodiments, the cancer is a gastrointestinal cancer. In some embodiments, the gastrointestinal is anal cancer, bile duct cancer, colon cancer, rectal cancer, esophageal cancer, gallbladder cancer, liver cancer, pancreatic cancer, small intestine cancer, or stomach cancer (gastric cancer).

In some embodiments, the cancer is colorectal cancer (CRC), pancreatic ductal adenocarcinoma (PDAC), cholangiocarcinoma cancer, appendiceal cancer, gastric cancer, esophageal cancer, non-small cell lung cancer (NSCLC), head and neck cancer, ovarian cancer, uterine cancer, acute myeloid leukemia (AML), or melanoma.

In some embodiments, the cancer is a gastrointestinal cancer. In some embodiments, the gastrointestinal is anal cancer, bile duct cancer, colon cancer, rectal cancer, esophageal cancer, gallbladder cancer, liver cancer, pancreatic cancer, small intestine cancer, or stomach cancer (gastric cancer).

### Dosing

In one aspect, the compositions described herein are used for the treatment of diseases and conditions described herein. In addition, a method for treating any of the diseases or conditions described herein in a subject in need of such treatment, involves administration of compositions in therapeutically effective amounts to said subject.

Dosages of compositions described herein can be determined by any suitable method. Maximum tolerated doses (MTD) and maximum response doses (MRD) for compound 1, or a pharmaceutically acceptable salt thereof can be determined via established animal and human experimental protocols as well as in the examples described herein. For example, toxicity and therapeutic efficacy of compound 1, or a pharmaceutically acceptable salt thereof, can be determined by standard pharmaceutical procedures in cell cultures or experimental animals, including, but not limited to, for determining the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. The data obtained from cell culture assays and animal studies can be used in formulating a range of dosage for use in human. The dosage of such compounds lies preferably within a range of circulating concentrations that include the ED50 with minimal toxicity. The dosage may vary within this range depending upon the dosage form employed and the route of administration utilized. Additional relative dosages, represented as a percent of maximal response or of maximum tolerated dose, are readily obtained via the protocols.

In some embodiments, the amount of a given formulation comprising compound 1, or a pharmaceutically acceptable salt thereof that corresponds to such an amount varies depending upon factors such as the particular salt or form, disease condition and its severity, the identity (e.g., age, weight, sex) of the subject or host in need of treatment, but can nevertheless be determined according to the particular circumstances surrounding the case, including, e.g., the specific agent being administered, the liquid formulation type, the condition being treated, and the subject or host being treated.

In some embodiments, encorafenib is administered in an amount that is between about 100 mg/day and 500 mg/day. In some embodiments, encorafenib is administered in an amount that is about 450 mg/day.

In some embodiments, dabrafenib is administered in an amount that is between about 50 mg/day and 200 mg/day. In some embodiments, dabrafenib is administered in an amount that is about 150 mg/day.

In some embodiments, panitumumab is administered in an amount that is 6 mg/kg. In some embodiments panitumumab is administered every 14 days. In some embodiments panitumumab is administered as an intravenous infusion over 60 minutes (<1000 mg) or 90 minutes (>1000 mg).

In some embodiments, compound 1, or a pharmaceutically acceptable salt thereof, is administered orally.

In some embodiments, compound 1, or a pharmaceutically acceptable salt thereof, is administered in an amount that is between about 25 mg/day and about 300 mg/day.

In some embodiments, compound 1, or a pharmaceutically acceptable salt thereof, is administered in an amount that is between 25 mg/day and 150 mg/day.

In some embodiments, compound 1, or a pharmaceutically acceptable salt thereof, is administered in an amount that is about 25 mg/day, about 50 mg/day, about 75 mg/day, about 100 mg/day, about 150 mg/day, about 175 mg/day, about 200 mg/day, about 225 mg/day, or about 250 mg/day.

In some embodiments, compound 1, or a pharmaceutically acceptable salt thereof, is administered in an amount that is about 25 mg/day, about 50 mg/day, about 100 mg/day, or about 150 mg/day.

In some embodiments, compound 1, or a pharmaceutically acceptable salt thereof, is administered in an amount between about 25 mg to about 300 mg twice a day, once a week (BID-QW).

In some embodiments, compound 1, or a pharmaceutically acceptable salt thereof, is administered in an amount that is between about 25 mg and about 250 mg twice a day, once a week (BID-QW).

In some embodiments, compound 1, or a pharmaceutically acceptable salt thereof, is administered in an amount that is between about 25 mg and about 200 mg twice a day, once a week (BID-QW).

In some embodiments, compound 1, or a pharmaceutically acceptable salt thereof, is administered in an amount that is between about 25 mg and about 150 mg twice a day, once a week (BID-QW).

In some embodiments, compound 1, or a pharmaceutically acceptable salt thereof, is administered in an amount that is between about 25 mg and about 100 mg twice a day, once a week (BID-QW).

In some embodiments, compound 1, or a pharmaceutically acceptable salt thereof, is administered in an amount that is between about 25 mg and about 50 mg twice a day, once a week (BID-QW).

In some embodiments, compound 1, or a pharmaceutically acceptable salt thereof, is administered in an amount that is between about 50 mg to about 300 mg twice a day, once a week (BID-QW).

In some embodiments, compound 1, or a pharmaceutically acceptable salt thereof, is administered in an amount that is between about 50 mg and about 250 mg twice a day, once a week (BID-QW).

In some embodiments, compound 1, or a pharmaceutically acceptable salt thereof, is administered in an amount that is between about 50 mg and about 200 mg twice a day, once a week (BID-QW).

In some embodiments, compound 1, or a pharmaceutically acceptable salt thereof, is administered in an amount that is between about 50 mg and about 150 mg twice a day, once a week (BID-QW).

In some embodiments, compound 1, or a pharmaceutically acceptable salt thereof, is administered in an amount that is between about 50 mg and about 100 mg twice a day, once a week (BID-QW).

In some embodiments, compound 1, or a pharmaceutically acceptable salt thereof, is administered in an amount that is between about 100 mg and about 300 mg twice a day, once a week (BID-QW).

In some embodiments, compound 1, or a pharmaceutically acceptable salt thereof, is administered in an amount that is between about 100 mg and about 250 mg twice a day, once a week (BID-QW).

In some embodiments, compound 1, or a pharmaceutically acceptable salt thereof, is administered in an amount that is between about 100 mg and about 200 mg twice a day, once a week (BID-QW).

In some embodiments, compound 1, or a pharmaceutically acceptable salt thereof, is administered in an amount that is between about 100 mg and about 150 mg twice a day, once a week (BID-QW).

In some embodiments, compound 1, or a pharmaceutically acceptable salt thereof, is administered in an amount that is between about 150 mg and about 300 mg twice a day, once a week (BID-QW).

In some embodiments, compound 1, or a pharmaceutically acceptable salt thereof, is administered in an amount that is between about 150 mg and about 250 mg twice a day, once a week (BID-QW).

In some embodiments, compound 1, or a pharmaceutically acceptable salt thereof, is administered in an amount that is between about 150 mg and about 200 mg twice a day, once a week (BID-QW).

In some embodiments, compound 1, or a pharmaceutically acceptable salt thereof, is administered in an amount that is between about 175 mg and about 300 mg twice a day, once a week (BID-QW).

In some embodiments, compound 1, or a pharmaceutically acceptable salt thereof, is administered in an amount that is between about 175 mg and about 250 mg twice a day, once a week (BID-QW).

In some embodiments, compound 1, or a pharmaceutically acceptable salt thereof, is administered in an amount that is between about 175 mg and about 200 mg twice a day, once a week (BID-QW).

In some embodiments, compound 1, or a pharmaceutically acceptable salt thereof, is administered in an amount that is between about 200 mg and about 300 mg twice a day, once a week (BID-QW).

In some embodiments, compound 1, or a pharmaceutically acceptable salt thereof, is administered in an amount that is between about 200 mg and about 250 mg twice a day, once a week (BID-QW).

In some embodiments, compound 1, or a pharmaceutically acceptable salt thereof, is administered in an amount that is between about 225 mg and about 300 mg twice a day, once a week (BID-QW).

In some embodiments, compound 1, or a pharmaceutically acceptable salt thereof, is administered in an amount that is between about 225 mg and about 250 mg twice a day, once a week (BID-QW).

In some embodiments, compound 1, or a pharmaceutically acceptable salt thereof, is administered in an amount that is about 250 mg once a day, once a week.

In some embodiments, compound 1, or a pharmaceutically acceptable salt thereof, is administered in an amount that is about 25 mg, 30 mg, 40 mg, 50 mg, about 60 mg, about 70 mg, about 75 mg, about 80 mg, about 90 mg, about 100 mg, about 110 mg, about 120 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 280 mg, about 290 mg, or about 300 mg.

### Administration

Administration of compound 1, or a pharmaceutically acceptable salt thereof, and combination partners described herein are at a dosage described herein or at other dose levels and compositions determined and contemplated by a medical practitioner. In certain embodiments, compound 1, or a pharmaceutically acceptable salt thereof, is administered for prophylactic and/or therapeutic treatments. In certain therapeutic applications, compound 1, or a pharmaceutically acceptable salt thereof, and combination partners described herein, are administered to a patient already suffering from a disease in an amount sufficient to cure the disease or at least partially arrest or ameliorate the symptoms. Amounts effective for this use depend on the age of the patient, severity of the disease, previous therapy, the patient's health status, weight, and response to the compositions, and the judgment of the treating physician. Therapeutically effective amounts are optionally determined by methods including, but not limited to, a dose escalation clinical trial.

In prophylactic applications, the compositions described herein are administered to a patient susceptible to or otherwise at risk of a particular disease, e.g., cancer. Such an amount is defined to be a “prophylactically effective amount or dose.” In this use, the precise amounts also depend on the patient's age, state of health, weight, and the like. When used in a patient, effective amounts for this use will depend on the risk or susceptibility of developing the particular disease, previous therapy, the patient's health status and response to the compositions, and the judgment of the treating physician.

In certain embodiments wherein the patient's condition does not improve, upon the doctor's discretion the administration of a composition described herein are administered chronically, that is, for an extended period of time, including throughout the duration of the patient's life in order to ameliorate or otherwise control or limit the symptoms of the patient's disease. In other embodiments, administration of a composition continues until complete or partial response of a disease.

In some embodiments, compound 1, or a pharmaceutically acceptable salt thereof, and combination partners described herein, are administered once a day. In some embodiments, compound 1, or a pharmaceutically acceptable salt thereof, and combination partners described herein are administered twice a day. In some embodiments, compound 1, or a pharmaceutically acceptable salt thereof, and combination partners described herein are administered three times a day.

In some embodiments, encorafenib is administered in an amount that is about 450 mg/day. In some embodiments, encorafenib is administered once daily. In some embodiments, encorafenib is administered twice daily. In some embodiments, encorafenib is administered three times daily.

In some embodiments, dabrafenib is administered once daily. In some embodiments, dabrafenib is administered twice daily. In some embodiments, dabrafenib is administered three times daily.

In some embodiments, compound 1, or a pharmaceutically acceptable salt thereof, and combination partners described herein are administered to a subject who is in a fasted state. A fasted state refers to a subject who has gone without food or fasted for a certain period of time. General fasting periods include at least 4 hours, at least 6 hours, at least 8 hours, at least 10 hours, at least 12 hours, at least 14 hours and at least 16 hours without food. In some embodiments, compound 1, or a pharmaceutically acceptable salt thereof, is administered to a subject who is in a fasted state for at least 8 hours. In other embodiments, compound 1, or a pharmaceutically acceptable salt thereof, and combination partners described herein, are administered to a subject who is in a fasted state for at least 10 hours. In yet other embodiments, compound 1, or a pharmaceutically acceptable salt thereof, and combination partners described herein, are administered to a subject who is in a fasted state for at least 12 hours. In other embodiments, compound 1, or a pharmaceutically acceptable salt thereof, and combination partners described herein, are administered to a subject who has fasted overnight.

In other embodiments, compound 1, or a pharmaceutically acceptable salt thereof, and combination partners described herein, are administered to a subject who is in a fed state. A fed state refers to a subject who has taken food or has had a meal. In certain embodiments, a composition is administered to a subject in a fed state 5 minutes post-meal, 10 minutes post-meal, 15 minutes post-meal, 20 minutes post-meal, 30 minutes post-meal, 40 minutes post-meal, 50 minutes post-meal, 1 hour post-meal, or 2 hours post-meal. In certain instances, compound 1, or a pharmaceutically acceptable salt thereof, is administered to a subject in a fed state 30 minutes post-meal. In other instances, compound 1, or a pharmaceutically acceptable salt thereof, and combination partners described herein, are administered to a subject in a fed state 1 hour post-meal. In yet further embodiments, compound 1, or a pharmaceutically acceptable salt thereof, is administered to a subject with food.

The length of a treatment cycle depends on the treatment being given. In some embodiments, the length of a treatment cycle ranges from two to six weeks. In some embodiments, the length of a treatment cycle ranges from three to six weeks. In some embodiments, the length of a treatment cycle ranges from three to four weeks. In some embodiments, the length of a treatment cycle is three weeks (or 21 days). In some embodiments, the length of a treatment cycle is four weeks (28 days). In some embodiments, the length of a treatment cycle is five weeks (35 days). In some embodiments, the length of a treatment cycle is 56 days. In some embodiments, a treatment cycle lasts one, two, three, four, or five weeks. In some embodiments, a treatment cycle lasts three weeks. In some embodiments, a treatment cycle lasts four weeks. In some embodiments, a treatment cycle lasts five weeks. The number of treatment doses scheduled within each cycle also varies depending on the drugs being given.

In some embodiments of a method of treating cancer, compound 1, or a pharmaceutically acceptable salt thereof, and combination partners described herein are administered in 21-day cycles. In some embodiments of a method of treating cancer, compound 1, or a pharmaceutically acceptable salt thereof, and combination partners described herein, are administered for multiple 21-day cycles. In some embodiments of a method of treating cancer, compound 1, or a pharmaceutically acceptable salt thereof, and combination partners described herein, are administered for at least one 21-day cycle. In some embodiments of a method of treating cancer, compound 1, or a pharmaceutically acceptable salt thereof, and combination partners described herein, are administered for at least two 21-day cycles. In some embodiments of a method of treating cancer, compound 1, or a pharmaceutically acceptable salt thereof, and combination partners described herein, are administered for at least three 21-day cycles. In some embodiments of a method of treating cancer, compound 1, or a pharmaceutically acceptable salt thereof, and combination partners described herein, are administered for at least four 21-day cycles. In some embodiments of a method of treating cancer, compound 1, or a pharmaceutically acceptable salt thereof, and combination partners described herein, are administered for at least five 21-day cycles. In some embodiments of a method of treating cancer, compound 1, or a pharmaceutically acceptable salt thereof, and combination partners described herein, are administered for at least six 21-day cycles.

In some embodiments of a method of treating cancer, compound 1, or a pharmaceutically acceptable salt thereof, and combination partners described herein are administered in 28-day cycles. In some embodiments of a method of treating cancer, compound 1, or a pharmaceutically acceptable salt thereof, and combination partners described herein, are administered for multiple 28-day cycles. In some embodiments of a method of treating cancer, compound 1, or a pharmaceutically acceptable salt thereof, and combination partners described herein, are administered for at least one 28-day cycle. In some embodiments of a method of treating cancer, compound 1, or a pharmaceutically acceptable salt thereof, and combination partners described herein, are administered for at least two 28-day cycles. In some embodiments of a method of treating cancer, compound 1, or a pharmaceutically acceptable salt thereof, and combination partners described herein, are administered for at least three 28-day cycles. In some embodiments of a method of treating cancer, compound 1, or a pharmaceutically acceptable salt thereof, and combination partners described herein, are administered for at least four 28-day cycles. In some embodiments of a method of treating cancer, compound 1, or a pharmaceutically acceptable salt thereof, and combination partners described herein, are administered for at least five 28-day cycles. In some embodiments of a method of treating cancer, compound 1, or a pharmaceutically acceptable salt thereof, and combination partners described herein, are administered for at least six 28-day cycles.

In some embodiments of a method of treating cancer, compound 1, or a pharmaceutically acceptable salt thereof, is administered on days 1-7 of each 28-day cycle. In some embodiments of a method of treating cancer, compound 1, or a pharmaceutically acceptable salt thereof, is administered on days 1-14 of each 28-day cycle. In some embodiments of a method of treating cancer, compound 1, or a pharmaceutically acceptable salt thereof, is administered on days 1-21 of each 28-day cycle. In some embodiments of a method of treating cancer, compound 1, or a pharmaceutically acceptable salt thereof, is administered on days 1-28 of each 28-day cycle.

In some embodiments of a method of treating cancer, compound 1, or a pharmaceutically acceptable salt thereof, is administered twice a day on day 1 of a 28-day cycle. In some embodiments of a method of treating cancer, compound 1, or a pharmaceutically acceptable salt thereof, is administered twice a day on day 8 of a 28-day cycle. In some embodiments of a method of treating cancer, compound 1, or a pharmaceutically acceptable salt thereof, is administered twice a day on day 15 of a 28-day cycle.

In some embodiments of a method of treating cancer, compound 1, or a pharmaceutically acceptable salt thereof, is administered twice a day on day 22 of a 28-day cycle. In some embodiments of a method of treating cancer, compound 1, or a pharmaceutically acceptable salt thereof, is not administered twice a day on day 22 of a 28-day cycle.

In some embodiments of a method of treating cancer, compound 1, or a pharmaceutically acceptable salt thereof, is administered twice a day on day 1, day 8, and day 15 of a 28-day cycle.

In some embodiments of a method of treating cancer, compound 1, or a pharmaceutically acceptable salt thereof, is not administered on days 2-7, days 9-14, days 16-21, days 23-28 of a 28-day cycle.

In some embodiments of a method of treating cancer, compound 1, or a pharmaceutically acceptable salt thereof, and combination partners described herein are administered in 35-day cycles. In some embodiments of a method of treating cancer, compound 1, or a pharmaceutically acceptable salt thereof, and combination partners described herein are administered for multiple 35-day cycles. In some embodiments of a method of treating cancer, compound 1, or a pharmaceutically acceptable salt thereof, and combination partners described herein are administered for at least one 35-day cycle. In some embodiments of a method of treating cancer, compound 1, or a pharmaceutically acceptable salt thereof, and combination partners described herein are administered for at least two 35-day cycle. In some embodiments of a method of treating cancer, compound 1, or a pharmaceutically acceptable salt thereof, and combination partners described herein are administered for at least three 35-day cycle. In some embodiments of a method of treating cancer, compound 1, or a pharmaceutically acceptable salt thereof, and combination partners described herein are administered for at least four 35-day cycle. In some embodiments of a method of treating cancer, compound 1, or a pharmaceutically acceptable salt thereof, and combination partners described herein are administered for at least five 35-day cycle. In some embodiments of a method of treating cancer, compound 1, or a pharmaceutically acceptable salt thereof, and combination partners described herein are administered for at least six 35-day cycle.

In some embodiments of a method of treating cancer, compound 1, or a pharmaceutically acceptable salt thereof, is administered on days 1-7 of each 35-day cycle. In some embodiments of a method of treating cancer, compound 1, or a pharmaceutically acceptable salt thereof, is administered on days 1-14 of each 35-day cycle. In some embodiments of a method of treating cancer, compound 1, or a pharmaceutically acceptable salt thereof, is administered on days 1-21 of each 35-day cycle. In some embodiments of a method of treating cancer, compound 1, or a pharmaceutically acceptable salt thereof, is administered on days 1-28 of each 35-day cycle. In some embodiments of a method of treating cancer, compound 1, or a pharmaceutically acceptable salt thereof, is administered on days 1-35 of each 35-day cycle.

In some embodiments of a method of treating cancer, compound 1, or a pharmaceutically acceptable salt thereof, is administered twice a day on day 1 of a 35-day cycle. In some embodiments of a method of treating cancer, compound 1, or a pharmaceutically acceptable salt thereof, is administered twice a day on day 8 of a 35-day cycle. In some embodiments of a method of treating cancer, compound 1, or a pharmaceutically acceptable salt thereof, is administered twice a day on day 15 of a 35-day cycle. In some embodiments of a method of treating cancer, compound 1, or a pharmaceutically acceptable salt thereof, is administered twice a day on day 22 of a 35-day cycle. In some embodiments of a method of treating cancer, compound 1, or a pharmaceutically acceptable salt thereof, is administered twice a day on day 29 of a 35-day cycle. In some embodiments of a method of treating cancer, compound 1, or a pharmaceutically acceptable salt thereof, is not administered twice a day on day 29 of a 35-day cycle.

In some embodiments of a method of treating a cancer, compound 1, or a pharmaceutically acceptable salt thereof, is administered twice a day on day 1, day 8, day 15, and day 22 of a 35-day cycle.

In some embodiments of a method of treating cancer, compound 1, or a pharmaceutically acceptable salt thereof, is not administered on days 2-7, days 9-14, days 16-21, days 23-28, and days 30-35 of a 28-day cycle.

### EXAMPLES

### Example 1: In-Vitro Viability Assay

Cells were plated at a density of 1,000 (RKO, HT-29, WiDr, MDST8) or 5,000 (LIM2405) cells per well in a 96-well plate (Corning #3903). Cells were allowed to adhere overnight, and compound was added in a matrix format using a HP Tecan D300e digital dispenser (Switzerland). Compound 1 was added in a 1:3 dilution series (8-point dose response) from bottom to top of plate (rows B-H) and encorafenib was added in a 1:3 dilution series (11-point dose response) from right to left (columns 2-11). Final DMSO concentration was normalized across the plate. Cell viability was assessed 5-days post-treatment using Promega CellTiter-Glo 3D Cell Viability Assay reagent (#G9683) according to manufacturer's protocol. Luminescence was assessed using a SpectraMax M3e (Molecular Devices, San Jose, CA) and combination benefit was assessed using the BLISS model in the Combenefit software (Cancer Research UK Cambridge Institute).

| | 1 | 2 | 3 | 4 | 5 | 6 | 7 | 8 | 9 | 10 | 11 | 12 |
|---|---|---|---|---|---|---|---|---|---|---|---|---|
| A | DMSO | 0.002y | 0.0039y | 0.0079y | 0.016y | 0.03y | 0.06y | 0.125y | 0.25y | 0.5y | y | DMSO |
| | only | | | | | | | | | | | only |
| B | 0.0014x | 0.0014x | 0.0014x | 0.0014x | 0.0014x | 0.0014x | 0.0014x | 0.0014x | 0.0014x | 0.0014x | 0.0014x | DMSO |
| | | 0.002y | 0.0039y | 0.0079y | 0.016y | 0.03y | 0.06y | 0.125y | 0.25y | 0.5y | y | only |
| C | 0.004x | 0.004x | 0.004x | 0.004x | 0.004x | 0.004x | 0.004x | 0.004x | 0.004x | 0.004x | 0.004x | DMSO |
| | | 0.002y | 0.0039y | 0.0079y | 0.016y | 0.03y | 0.06y | 0.125y | 0.25y | 0.5y | y | only |
| D | 0.012x | 0.012x | 0.012x | 0.012x | 0.012x | 0.012x | 0.012x | 0.012x | 0.012x | 0.012x | 0.012x | DMSO |
| | | 0.002y | 0.0039y | 0.0079y | 0.016y | 0.03y | 0.06y | 0.125y | 0.25y | 0.5y | y | only |
| E | 0.037x | 0.037x | 0.037x | 0.037x | 0.037x | 0.037x | 0.037x | 0.037x | 0.037x | 0.037x | 0.037x | DMSO |
| | | 0.002y | 0.0039y | 0.0079y | 0.016y | 0.03y | 0.06y | 0.125y | 0.25y | 0.5y | y | only |
| F | 0.11x | 0.11x | 0.11x | 0.11x | 0.11x | 0.11x | 0.11x | 0.11x | 0.11x | 0.11x | 0.11x | DMSO |
| | | 0.002y | 0.0039y | 0.0079y | 0.016y | 0.03y | 0.06y | 0.125y | 0.25y | 0.5y | y | only |
| G | 0.33x | 0.33x | 0.33x | 0.33x | 0.33x | 0.33x | 0.33x | 0.33x | 0.33x | 0.33x | 0.33x | DMSO |
| | | 0.002y | 0.0039y | 0.0079y | 0.016y | 0.03y | 0.06y | 0.125y | 0.25y | 0.5y | y | only |
| H | x | x | x | x | x | x | x | x | x | x | x | DMSO |
| | | 0.002y | 0.0039y | 0.0079y | 0.016y | 0.03y | 0.06y | 0.125y | 0.25y | 0.5y | y | only |

x: compound 1
y: encorafenib

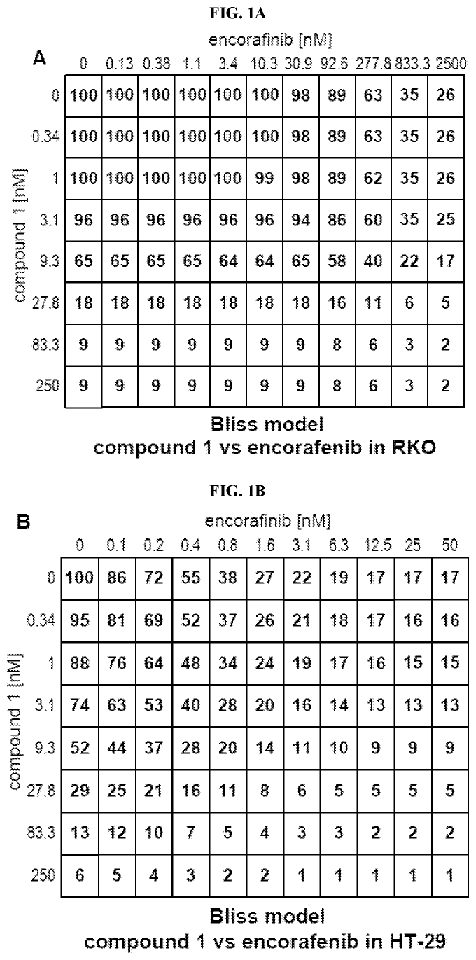

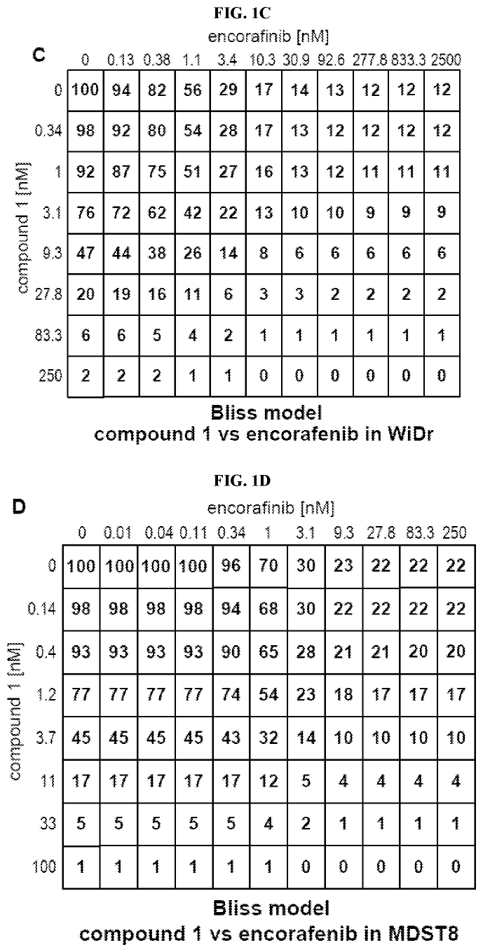

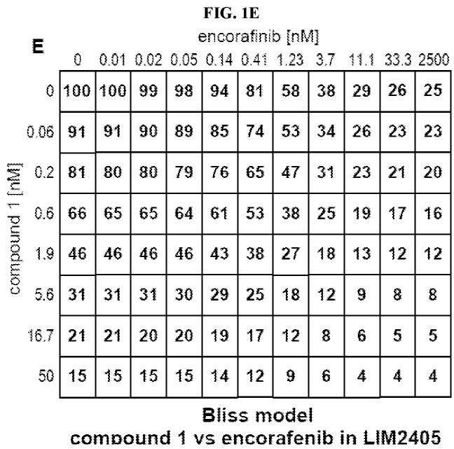

RKO cells (FIG. 1A), HT-29 cells (FIG. 1B), WiDr cells (FIG. 1C), MDST8 cells (FIG. 1D), and LIM2405 (FIG. 1E) were treated with a dilution matrix of compound 1 versus encorafenib in 3D cell viability assays. Cell viability, as expressed as a percentage of viable cells relative to vehicle treated control, is shown in the matrix. Compound 1 and encorafenib demonstrate combination benefit in vitro in cell lines harboring a BRAF^{V600E} mutation.

### Example 2: In-Vivo Assay

### Vehicle/Control Article

The vehicle/control article, 0.5% Methyl Cellulose & 0.1% Tween 80 or 100 mM acetic acid in deionized water with pH adjustment to 4.8-5.0, was prepared and stored under ambient conditions throughout the study period.

### Formulation of Test Article

The test article compound 1 was freshly prepared in vehicle of 0.5% Methyl Cellulose & 0.1% Tween 80 weekly and stored under ambient conditions. The combination agent, encorafenib, was freshly prepared in vehicle of 0.5% CMC and 0.5% Tween 80 weekly and stored at 2-8° C.

### Animals

Female Balb/c nude mice were purchased from the Beijing Vital River Laboratory Animal Technology Co., Ltd. Mice were hosted at special pathogen-free (SPF) environment of vivarium facility and acclimated to their new environment for at least 3 days prior to initiation of any experiments. Mice were between 6-8 weeks of age at the time of implantation.

All procedures related to animal handling, care, and treatment in this study were performed according to the protocols and guidelines approved by the Institutional Animal Care and Use Committee (IACUC) of GenenDesign and WuXi AppTec. Animal facility and program is operated under the standard of Guide for the Care and Use of Laboratory Animals (NRC, 2011) and accredited by the Association for Assessment and Accreditation of Laboratory Animal Care (AAALAC). Specifically, all portions of this study performed at GenenDesign and WuXi AppTec adhered to the study protocols reviewed and approved by IACUC and applicable standard operating procedures (SOPs).

### Preparation of Xenograft Model

RKO is a human CRC tumor cell line that harbors a BRAF^{V600E} mutation. The RKO cell line was purchased from the American Type Culture Collection (ATCC® CRL-2577™). RKO cells were cultured in medium containing EMEM plus 10% Fetal Bovine Serum (FBS) supplemented with non-essential amino acids at 37° C. in an atmosphere of 5% CO₂ in air. RKO cells in 200 μL cell suspensions containing 2×10⁶ cells mixed with 50% Matrigel were implanted into mice subcutaneously. When tumor volumes reached a mean of 200 mm³, tumor-bearing mice were randomized into different groups with 8 mice in each group and treatment started on the day of randomization.

WiDr is a human CRC tumor cell line that harbors a BRAF^{V600E} mutation. The WiDr cell line was purchased from the European Collection of Authenticated Cell Cultures (ECACC, 85111501). WiDr cells were cultured in medium containing EMEM (EBSS) plus 10% Fetal Bovine Serum (FBS), 2 mM Glutamine, and supplemented with 1% non-essential amino acids (NEAA) at 37° C. in an atmosphere of 5% CO₂ in air. WiDr cells in 200 μL cell suspensions containing 5×10⁶ cells mixed with 50% Matrigel were implanted into mice subcutaneously. When tumor volumes reached a mean of 190 mm³, tumor-bearing mice were randomized into different groups with 8 mice in each group and treatment started on the day of randomization.

### Treatment

Mice were dosed by oral administration of vehicle control solution, compound 1, or encorafenib in monotherapy treatment groups. Mice were dosed by oral administration of combination, including compound 1 with encorafenib. The dosing volume was 5 mL/kg for each compound and interval of BID regimen was 8 hours. In the combination of compound 1 with encorafenib, compound 1 was dosed at one-hour post encorafenib dose. In addition to regular food and water supply, DietGel (ClearH2O, US) was added in cages where at least two mice showed >10% BWL. The study was terminated at the end of 4-week treatment or when tumor volume in vehicle control group reached 2,000 mm³.

### Results

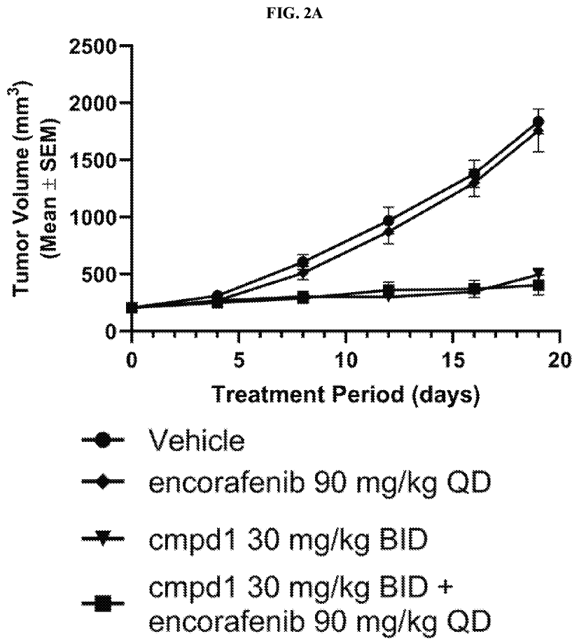

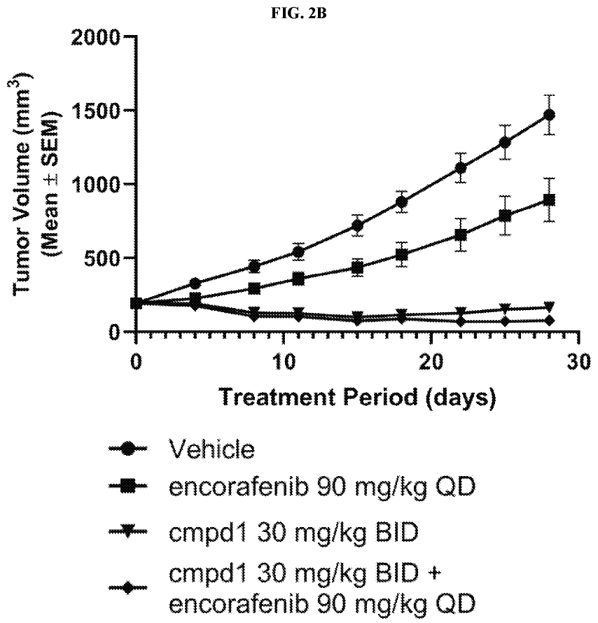

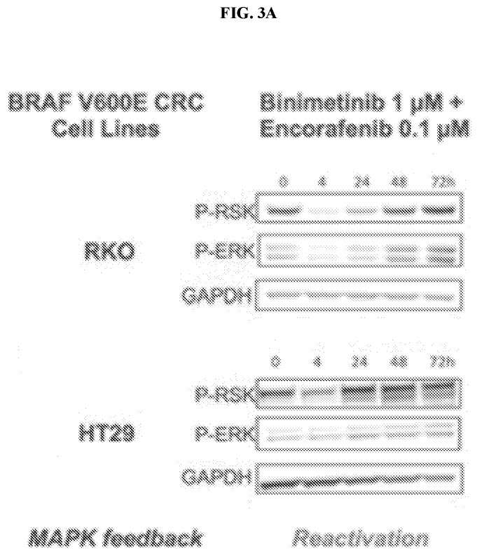

Compound 1 and encorafenib demonstrate combination benefit in vivo in encorafenib refractory-BRAFV^{600E} CDX models. Tumor growth curves for (FIG. 2A) RKO and (FIG. 2B) WiDr CDX models.

### Example 3: Treatment of BRAF V600E Mutant CRC Cell Lines (RKO and HT-29) with Encorafenib in Combinations with MEK and ERK Inhibitors

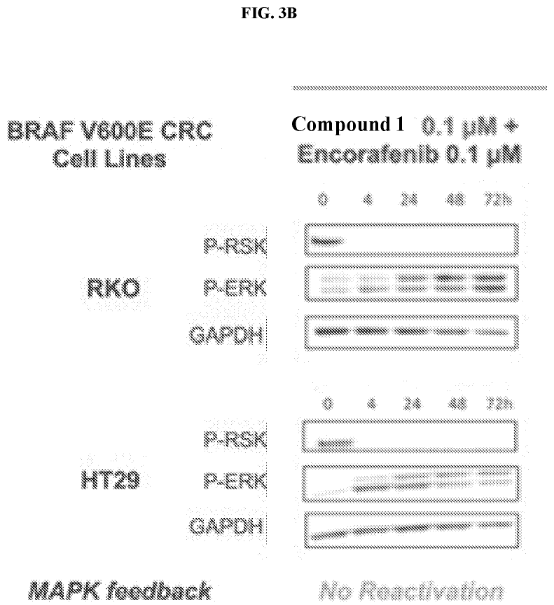

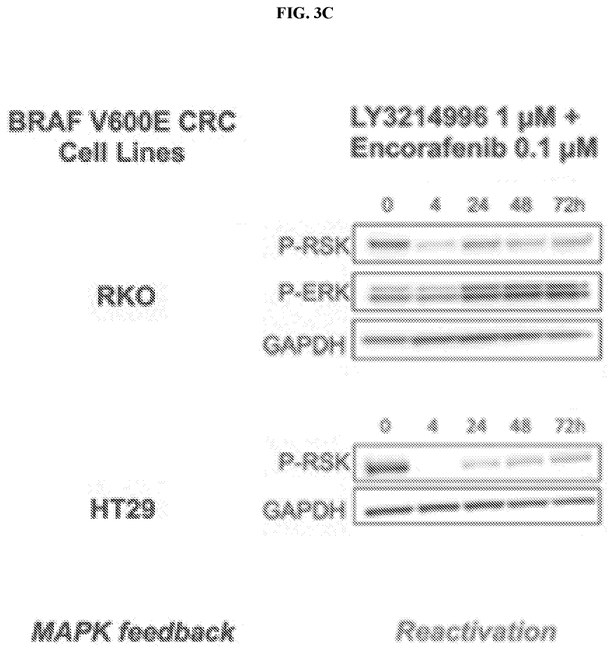

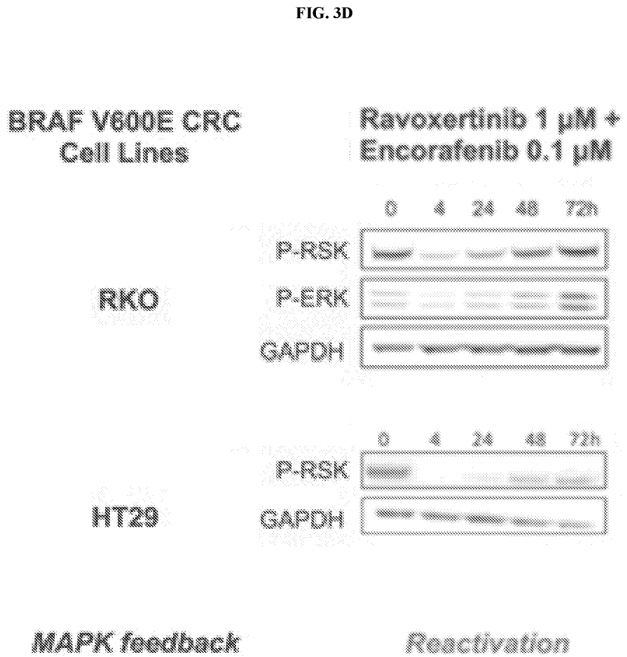

Treatment of two BRAF V600E mutant CRC cell lines, RKO and HT-29, with encorafenib in combination with the MEK inhibitor binimetinib, the ERK inhibitor Compound 1, the ERK inhibitor LY3214996, and the ERK inhibitor ravoxertinib. The Western blot gels depict phosphorylation of RSK (P-RSK) and ERK (P-ERK). Higher levels of phosphorylation are depicted by higher (i.e., darker) band intensity. Total GAPDH protein (GAPDH) serves as a loading control. ERK signaling activity is represented by the phosphorylation state of RSK (P-RSK), which is a downstream target of ERK. The column values indicate the duration of compound incubation of up to 72 hours. FIG. 3A-FIG. 3D.

In BRAF V600E colorectal cell lines, Compound 1 blocked the RAS/MAPK pathway feedback reactivation observed with MEK or other ERK plus BRAF inhibitor combinations at one-tenth the concentration used for the MEK and other ERK inhibitors. These results provide further support that inhibition of ERK by Compound 1 may lead to a more complete and durable blockade of the RAS/MAPK pathway relative to other inhibitors of ERK or MEK, either alone or in combination.

1. A method of treating cancer in a subject in need thereof, the method comprising: administering to the subject in need thereof a therapeutically effective amount of
(i) compound 1: 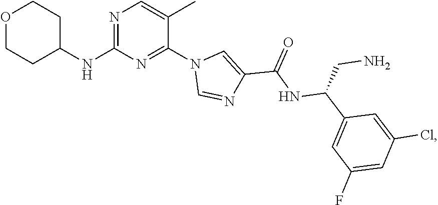 [SMILES: Cc1cnc(NC2CCOCC2)nc1-n1cnc(C(=O)N[C@H](CN)c2cc(F)cc(Cl)c2)c1]
or a pharmaceutically acceptable salt thereof, and
(ii) a BRAF inhibitor that is encorafenib or dabrafenib.

2. The method of claim 1, wherein the BRAF inhibitor is encorafenib.

3. The method of claim 2, wherein encorafenib is administered in an amount that is about 450 mg/day.

4. The method of claim 1, wherein the BRAF inhibitor is dabrafenib.

5. The method of claim 4, wherein dabrafenib is administered in an amount that is about 150 mg/day.

6. The method of any one of claims 1-5, wherein the method further comprises administering panitumumab.

7. The method of claim 6, wherein panitumumab is administered in an amount that is about 6 mg/kg.

8. A method of treating a cancer in a subject in need thereof, the method comprising: administering to the subject in need thereof a therapeutically effective amount of
(i) compound 1: 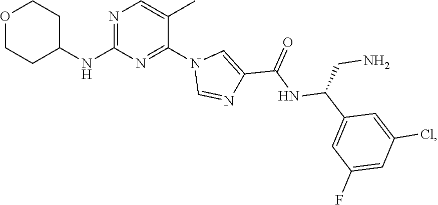 [SMILES: Cc1cnc(NC2CCOCC2)nc1-n1cnc(C(=O)N[C@H](CN)c2cc(F)cc(Cl)c2)c1]
or a pharmaceutically acceptable salt thereof;
(ii) a BRAF inhibitor that is encorafenib or dabrafenib; and
(iii) panitumumab.

9. The method of claim 8, wherein the BRAF inhibitor is encorafenib.

10. The method of claim 9, wherein encorafenib is administered in an amount that is about 450 mg/day.

11. The method of claim 8, wherein the BRAF inhibitor is dabrafenib.

12. The method of claim 11, wherein dabrafenib is administered in an amount that is about 150 mg/day.

13. The method of any one of claims 8-12, wherein panitumumab is administered in an amount that is 6 mg/kg.

14. A method of treating a cancer in a subject in need thereof, the method comprising: administering to the subject in need thereof a therapeutically effective amount of
(i) compound 1: 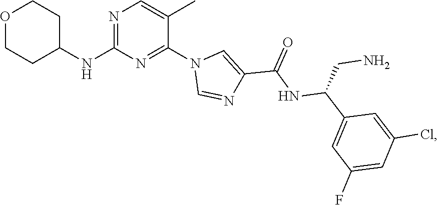 [SMILES: Cc1cnc(NC2CCOCC2)nc1-n1cnc(C(=O)N[C@H](CN)c2cc(F)cc(Cl)c2)c1]
or a pharmaceutically acceptable salt thereof; and
(ii) panitumumab.

15. The method of claim 14, wherein panitumumab is administered in an amount that is 6 mg/kg.

16. The method of any one of claims 1-15, wherein the pharmaceutically acceptable salt of compound 1 is the mandelic acid salt.

17. The method of any one of claims 1-16, wherein the cancer is a mitogen-activated protein kinase (MAPK) pathway driven cancer.

18. The method of any one of claims 1-16, wherein the cancer is a BRAF-driven cancer, HRAS-driven cancer, or aNRAS-driven cancer.

19. The method of any one of claims 1-16, wherein the cancer comprises at least one cancer cell driven by deregulated ERK.

20. The method of any one of claims 1-16, wherein the cancer has at least one mutation in RAS.

21. The method of any one of claims 1-16, wherein the cancer has at least one mutation in RAF.

22. The method of any one of claims 1-16, wherein the cancer has at least one mutation in MEK.

23. The method of any one of claims 1-16, wherein the cancer has a G12C KRAS mutation.

24. The method of any one of claims 1-16, wherein the cancer has a G12D KRAS mutation.

25. The method of any one of claims 1-16, wherein the cancer has a G12S KRAS mutation.

26. The method of any one of claims 1-16, wherein the cancer has a G12V KRAS mutation.

27. The method of any one of claims 1-16, wherein the cancer has a G13D KRAS mutation.

28. The method of any one of claims 1-16, wherein the cancer has a Q16H KRAS mutation.

29. The method of any one of claims 1-16, wherein the cancer has a Q16K KRAS mutation.

30. The method of any one of claims 1-16, wherein the cancer has a Q61RNRAS mutation.

31. The method of any one of claims 1-16, wherein the cancer is a BRAF V600E or V600K mutant tumor.

32. The method of any one of claims 1-16, wherein the cancer is a MAPKm/MAPKi-naïve pan cancer.

33. The method of any one of claims 1-16, wherein the cancer comprises one or more EGFR mutation selected from the group consisting of EGFR gene copy gain, EGFR gene amplification, chromosome 7 polysomy, L858R, exon 19 deletions/insertions, L861Q, G719C, G719S, G719A, V765A, T783A, exon 20 insertions, EGFR splice variants (Viii, Vvi, and Vii), A289D, A289T, A289V, G598A, G598V, T790M, and C797S.

34. The method of any one of claims 1-16, wherein the cancer comprises one or more EGFR mutation selected from the group consisting of L858R, exon 19 deletion, and T790M.

35. The method of any one of claims 1-34, wherein the cancer is a solid tumor.

36. The method of any one of claims 1-35, wherein the cancer is non-small cell lung cancer (NSCLC), melanoma, pancreatic cancer, salivary gland tumor, thyroid cancer, colorectal cancer (CRC), or esophageal cancer.

37. The method of any one of claims 1-35, wherein the cancer is non-small cell lung cancer (NSCLC).

38. The method of claim 37, wherein the NSCLC is an EGFR mutant NSCLC.

39. The method of claim 37, wherein the NSCLC is a KRAS G12C mutant NSCLC.

40. The method of claim 37, wherein the NSCLC is a KRAS G12D mutant NSCLC.

41. The method of claim 37, wherein the NSCLC is a KRAS G12S mutant NSCLC.

42. The method of claim 37, wherein the NSCLC is a KRAS G12V mutant NSCLC.

43. The method of claim 37, wherein the NSCLC is a KRAS G13D mutant NSCLC.

44. The method of claim 37, wherein the NSCLC is a KRAS Q61H mutant NSCLC.

45. The method of claim 37, wherein the NSCLC is a KRAS Q61K mutant NSCLC.

46. The method of claim 37, wherein the NSCLC is a NRAS Q61R mutant NSCLC.

47. The method of claim 37, wherein the cancer is a MAPKm/MAPKi-naïve NSCLC.

48. The method of claim 37, wherein the cancer is a BRAFi-treated V600 NSCLC.

49. The method of claim 37, wherein the cancer is a KRAS-treated G12C NSCLC.

50. The method of claim 37, wherein the cancer is a KRAS-treated G12D NSCLC.

51. The method of claim 37, wherein the cancer is a KRAS-treated G12S NSCLC.

52. The method of claim 37, wherein the cancer is a KRAS-treated G12V NSCLC.

53. The method of claim 37, wherein the cancer is a KRAS-treated G13D NSCLC.

54. The method of claim 37, wherein the cancer is a KRAS-treated Q61H NSCLC.

55. The method of claim 37, wherein the cancer is a KRAS-treated Q61KNSCLC.

56. The method of claim 37, wherein the cancer is aNRAS-treated Q61RNSCLC.

57. The method of any one of claims 1-35, wherein the cancer is pancreatic cancer.

58. The method of claim 57, wherein the cancer is a MAPKm/MAPKi-naïve pancreatic cancer.

59. The method of any one of claims 1-35, wherein the cancer is melanoma.

60. The method of claim 59, wherein the melanoma is a BRAF V600E or V600K mutant tumor.

61. The method of claim 59, wherein the cancer is a BRAFi-treated V600 melanoma.

62. The method of any one of claims 1-35, wherein the cancer is salivary gland tumor.

63. The method of any one of claims 1-35, wherein the cancer is thyroid cancer.

64. The method of any one of claims 1-35, wherein the cancer is colorectal cancer (CRC).

65. The method of claim 64, wherein the CRC is a BRAF V600E CRC.

66. The method of claim 64, wherein the CRC is a KRAS mutant CRC.

67. The method of claim 66, wherein the CRC is a KRAS G12C mutant CRC.

68. The method of claim 66, wherein the CRC is a KRAS G12D mutant CRC.

69. The method of claim 66, wherein the CRC is a KRAS G12S mutant CRC.

70. The method of claim 66, wherein the CRC is a KRAS G12V mutant CRC.

71. The method of claim 66, wherein the CRC is a KRAS G13D mutant CRC.

72. The method of claim 66, wherein the CRC is a KRAS Q61H mutant CRC.

73. The method of claim 66, wherein the CRC is a KRAS Q61K mutant CRC.

74. The method of claim 64, wherein the CRC is aNRAS mutant CRC.

75. The method of claim 74, wherein the CRC is aNRAS Q61R mutant CRC.

76. The method of any one of claims 1-35, wherein the cancer is esophageal cancer.

77. The method of any one of claims 1-76, wherein compound 1, or a pharmaceutically acceptable salt thereof, is administered in an amount that is between about 25 mg/day and about 300 mg/day.

78. The method of any one of claims 1-77, wherein compound 1, or a pharmaceutically acceptable salt thereof, is administered in an amount that is between 25 mg/day and 150 mg/day.

79. The method of any one of claims 1-78, wherein compound 1, or a pharmaceutically acceptable salt thereof, is administered in an amount that is about 25 mg/day, about 50 mg/day, about 75 mg/day, about 100 mg/day, about 150 mg/day, about 175 mg/day, about 200 mg/day, about 225 mg/day, or about 250 mg/day.

80. The method of any one of claims 1-79, wherein compound 1, or a pharmaceutically acceptable salt thereof, is administered in an amount that is about 25 mg/day, about 50 mg/day, about 100 mg/day, or about 150 mg/day.

81. The method of any one of claims 1-76, wherein compound 1, or a pharmaceutically acceptable salt thereof, is administered in an amount that is about 250 mg/day.

82. The method of any one of claims 1-81, wherein compound 1, or a pharmaceutically acceptable salt thereof, is administered once a day (QD).

83. The method of any one of claims 1-81, wherein compound 1, or a pharmaceutically acceptable salt thereof, is administered twice a day (BID).

84. The method of any one of claims 1-81, wherein compound 1, or a pharmaceutically acceptable salt thereof, is administered three times a day (TID).

85. The method of any one of claims 1-84, wherein compound 1, or a pharmaceutically acceptable salt thereof, is administered once a week.

86. The method of any one of claims 1-84, wherein compound 1, or a pharmaceutically acceptable salt thereof, is administered in an amount that is between about 50 mg once a week and about 400 mg once a week.

87. The method of any one of claims 1-84, wherein compound 1, or a pharmaceutically acceptable salt thereof, is administered twice a week.

88. The method of any one of claims 1-84, wherein compound 1, or a pharmaceutically acceptable salt thereof, is administered in an amount that is between about 50 mg twice a week and about 400 mg twice a week.

89. The method of any one of claims 1-88, wherein compound 1, or a pharmaceutically acceptable salt thereof, is administered for at least one 28-day cycle.

90. The method of any one of claims 1-89, wherein compound 1, or a pharmaceutically acceptable salt thereof, is administered on day 1, day 8, day 15, and day 22 of a 28-day cycle.

91. The method of any one of claims 1-89, wherein compound 1, or a pharmaceutically acceptable salt thereof, is administered on day 1, day 8, day 15 of a 28-day cycle.

92. The method of any one of claims 1-88, wherein compound 1, or a pharmaceutically acceptable salt thereof, is administered for at least one 21-day cycle.

93. The method of any one of claims 1-92, wherein compound 1, or a pharmaceutically acceptable salt thereof, is administered orally.
